(19) United States
(12) Patent Application Publication
(10) Pub. No.: US 2006/0045928 A1
(43) Pub. Date: Mar. 2, 2006

(54) Composition containing herbal medicine component for promoting absorption and production method thereof

(76) Inventor: Seiri Oshiro, Okinawa (JP)

Correspondence Address: Patrick G. Burns, Esq.;GREER, BURNS & CRAIN, LTD., Suite 2500, 300 South Wacker Dr., Chicago, IL, 60606, US

(21) Appl. No.: 11/152,403

(22) Filed: Jun. 14, 2005

(30) Foreign Application Priority Data
Aug. 31, 2004 (JP) 2004-252417

(51) Int. Cl. A61K 36/9066; A61K 36/58; A01N 65/00

(52) U.S. Cl. 424/756; 424/761

(57) ABSTRACT

To make diacylglycerol obtained by adding an emulsifier contain both an effective oil-soluble component and an effective water-soluble component which are contained in herbal medicines, a herbal medicine component for promoting the absorption of the oil-soluble component in the small intestine and a production method thereof are objects of the present invention. The powder of the herbal medicines is added to solvent oil selected from diacylglycerol and/or triacylglycerol cooking oil to which lecithin and/or a W/O type emulsifier are added, is heated and an herbal medicine component is extracted or dissolved. By such a method, drugs and healthy food which contain all effective oil-soluble and water-soluble components contained in the herbal medicines and which promote the absorption of the oil-soluble components in the small intestine can be produced.

### BACKGROUND OF THE INVENTION

1. Field of the Invention

The present invention relates to making diacylglycerol obtained by adding an emulsifier containing an effective oil-soluble component and an effective water-soluble component-containing in herbal medicines, a composition containing a herbal medicine component for promoting the absorption into the small intestine of the oil-soluble component and a production method thereof.

2. Description of the Prior Art

There is an oil-soluble component and a water-soluble component in an effective component of herbal medicines. A method for extracting an effective component in high density requires a device. As one of such devices, conventionally a method for boiling herbal medicines in water was used in Japan, China and the East. However, only the water-soluble component is extracted by this method, the oil-soluble component is not efficiently extracted and is discarded together with boiled grounds.

As methods for extracting the oil-soluble component from herbal medicines, there is a method of soaking herbal medicines in sake, vinegar or cooking oil and a method of frying herbal medicines in cooking oil. However, it is difficult to extract an oil-soluble component with sufficient density by such conventional methods.

A part of an oil-soluble component, such as curcumin and the like contained in the root/stem of fall curcuma (scientific name: curcuma longa) can be simply extracted with high density using ethanol or acetic acid. However, such ethanol or acetic acid cannot be eaten as food without further processing. Relating to a method for extracting curcumin with high density by soaking and heating the root/stem of curcuma longa in cooking oil instead of ethanol or acetic acid, there are the following inventions invented by this inventor and others.

According to a prior art search, Patent References disclose the following method for extracting an oil-soluble component in cooking oil. For example, there is a “Production Method of Flavored Oil by Far Infrared Rays (see Patent Reference 1). In this method, flavored oil is extracted by soaking spices and/or flavored plants in cooking oil and applying far infrared rays to them.

There is also a “Cooking Oil Containing Curcuma and Production method thereof” by the current inventor (see Patent Reference 2). In this method, oil-soluble components, such as curcumin, and refined oil are extracted by soaking the root/stem powder of curcuma longa and applying far infrared rays, and the bitter component of curcuma is eliminated.

Furthermore, there is a “Diacylglycerol Solution Containing Curcuma and Production Method thereof” also by the current inventor (see Patent Reference 3). In this method, the extraction density of curcumin is improved by using diacylglycerol as a solvent, compared with using ordinary cooking oil as a solvent. There is also a “Method for Extracting Curcumin from Curcuma” (Patent Reference 4). In this method, curcumin is absorbed and enveloped by cyclodextrin after curcumin whose peculiar fragrance, offensive odor and bitter taste are definitely eliminated by ultra-critical carbon dioxide, is extracted. However, since the method of Patent Reference 4 is a method for enveloping pure curcumin whose bitter taste and the like are eliminated, with cyclodextrin, the method has no relationship to the present invention.

The lecithin of a bile component has a function to emulsify oils and fats and promote its absorption (see Non-Patent Reference 1). Patent References 1, 2, 3 and 4 are Japanese Patent Application Nos. Hei05-316989, 2001-086931, 2003-137799 and Hei06-009479, respectively. Non-Patent Reference 1 is “Standard Physiology Second edition”, Medical-shoin, p 603-604 (1922).

The method of extracting an oil-soluble component in cooking oil has the following problems.

The first problem is that although there are an oil-soluble component and a water-soluble component in the effective component of herbal medicines, it is only the oil-soluble component that is extracted by cooking oil. Although the oil-soluble component can be extracted with a high density, the water-soluble component cannot be extracted. The first object of the present invention is to extract a water-soluble component as well as an oil-soluble component in cooking oil.

The second problem is how to promote the absorption into the small intestine of an oil-soluble component. Although Patent References 1 through 3 all relate to a method for extracting an oil-soluble component with high density, none of them promotes the absorption into the small intestine of an oil-soluble component.

However, it is generally known that if an oil-soluble component is extracted in cooking oil and is fried with oil, its absorption is promoted. For example, if carrot is fried with oil, the absorption rate of the β-carotene of an oil-soluble component is improved approximately 5 to 10 times, compared with eating raw carrot. Therefore, it is admitted that the absorption is promoted by simply dissolving an oil-soluble component in cooking oil.

The object of ingesting herbal medicines or healthy food is to prevent disease, to cure disease and to sustain health. However, there are many people who are not sufficiently well to take these medicines, discharge little bile and cannot digest/absorb them sufficiently well. If there is little bile, the small intestine cannot effectively absorb an oil-soluble component.

As disclosed by Non-Patent Reference 1, a mechanism for absorbing an oil-soluble component is as follows. Firstly, the lecithin of a bile component discharged by the small intestine emulsifies an oil-soluble component and then the small intestine absorbs the oil-soluble component. Therefore, by mixing lecithin with an oil-soluble component in advance, even in the case of a person who does not discharge a sufficient amount of bile the mixture of lecithin and a solvent can effectively emulsify the oil-soluble component and promoting its absorption.

The second object of the present invention is to promote the absorption of an oil-soluble component even in the absence of sufficient discharge of bile, by dissolving in advance lecithin in oil, which is a solvent.

### SUMMARY OF THE INVENTION

In the preferred embodiment of the present invention, curcuma longa and curcumin (C21H2006) several percent of which is contained in the dried root/stem of curcuma longa are used as herbal medicines and an effective oil-soluble component-containing in the curcuma longa, respectively. Curcuma longa also contains several percent of refined oil in addition to curcumin as an oil-soluble component. It is said that the refined oil also contains several tens to several hundreds of various components, which are synergistic. Curcuma longa also contains water-soluble bitter components besides the above-mentioned curcumin, the refined oil and the like. In Okinawa prefecture, people have traditionally eaten a very bitter preparation obtained by grating raw curcuma longa. Generally curcuma longa is boiled and the water used to boil it drunk. This is because the water-soluble bitter component has a pharmacological effect as a digestive.

As a method for extracting the oil-soluble component of herbal medicines in cooking oil with high density, there is a method for extracting curcumin using olive oil as a solvent, disclosed in Patent Reference 2 by the current inventor, and a method for extracting curcumin using diacylglycerol as a solvent, disclosed in Patent Reference 3.

In these methods, it is verified that if diacylglycerol is used, curcumin can be extracted with a higher density, than is possible with olive oil.

Therefore, in the present invention, diacylglycerol is used as a solvent for extracting an herbal medicine component. However, if an herbal medicine component with high density is not necessarily required another cooking oil is substituted.

Comparison between the amount of curcumin extracted using olive oil and diacylglycerol as a solvent disclosed in Patent References 2 and 3, respectively, and the amount of curcumin extracted by boiling the root/stem powder of curcuma longa in water is shown in FIG. 1.

As shown in FIG. 1, large amounts of curcumin can be extracted 7.55 mg/ml and 11.22 mg/ml using olive oil and diacylglycerol, respectively. However, curcumin can barely be extracted by the conventional boiling of curcuma tea made from curcuma longa.

The first problem can be solved by adding the root/stem powder of curcuma longa to diacylglycerol to which a W/o (water/oil) type emulsifier is added and/or triacylglycerol cooking oil, heating them and extracting a curcuma longa component. By this method, the W/O type emulsifier absorbs and extracts the water-soluble bitter component of the curcuma longa.

In order to confirm that by adding a W/O type emulsifier to olive oil, a water-soluble component can be extracted the following experiment was conducted.

1. Heavy brown liquid was obtained by adding 20 g of spring curcuma root/stem powder containing a large amount of a bitter component to 100 g of olive oil and heating them at 90° C. for half an hour.

2. Heavy brown liquid was obtained by adding 5 g of a W/O type emulsifier, glyceric fatty acid ester to 100 g of olive oil, adding 20 g of spring curcuma longa and heating them at 90° C. for half an hour.

In FIG. 2, the extraction/non-extraction of a water-soluble component is compared between a case where no W/O type emulsifier is added and a case where a W/O type emulsifier is added. If there is a bitter taste, the extraction of a water-soluble component is considered to have occurred.

The second problem can be solved by extracting a curcuma longa component in diacylglycerol to which lecithin and/or triacylglycerol cooking oil is added in advance. Lecithin is a bile component, and promotes the absorption of oil in the small intestine by emulsifying oil. By adding lecithin to oil in advance as a solvent and extracting an oil-soluble component, the oil-soluble component and lecithin are mixed, the added lecithin compensates for insufficient lecithin in bile and insufficient discharge of bile, and the oil-soluble component can be sufficiently emulsified and absorbed. The present invention is based on the above presented knowledge.

The respective solutions of the first and second problems can be summarized as follows. By adding the powder of herbal medicines to diacylglycerol to which lecithin and/or a W/O type emulsifier are added, and/or an oil selected from triacylglycerol cooking oil as a solvent, heating the resulting mixture and extracting or dissolving herbal medicines, both an oil-soluble component and a water-soluble component can be dissolved, and accordingly, a composition containing the herbal medicine component for promoting the absorption of the oil-soluble component into the small intestine can be obtained.

Diacylglycerol as used in this specification is an intermediate product obtained in the process in which the main component of cooking oil, triacylglycerol is decomposed into monoacylglycerol and fatty acid by a fat decomposing enzyme, lipase. In the decomposition process, diacylglycerol and monoacylglycerol are also mixed and produced. In triacylglycerol, three units of fatty acid are ester-connected to glycerol.

However, in diacylglycerol, two units of fatty acid are ester-connected to glycerol, and there are two ismers each with a 1,2-structure or 1,3-structure, in diacylglycerol, which exist in nature in the ratio of 3 to 7. Several percent of diacylglycerol exists in most of animal/plant cooking oil. Olive oil and soybean oil contain 5.5% and 1.0%, respectively, of diacylglycerol. In monoacylglycerol, one unit of fatty acid is connected to glycerol. Currently, since diacylglycerol prevents obesity, a product which consists of 80% diacylglycerol is sold as cooking oil.

Although in the present invention, cooking oil with 80% of diacylglycerol is used, monoacylglycerol and triacylglycerol can also be mixed with diacylglycerol.

Triacylglycerol is the main component of cooking oil and occupies approximately 80 to 90% of cooking oil. Cooking oil includes plant oils, such as soybean oil, olive oil, sesame oil, rape oil, corn oil, palm oil, peanut oil, germ oil, perilla oil and the like, and animal oils, such as tallow, lard oil, cod-liver oil, fish oil, egg oil, crow bone egg oil, snake oil, EPA (eicosapentaenoic acid), DHA (docosahexaenoic acid) and the like, and cooking oil obtained by processing such oil, such as squalane, squalene and the like. In the present invention, such cooking oil is collectively called triacylglycerol cooking oil. As a solvent for extracting an oil-soluble component, oil selected from single diacylglycerol and/or triacylglycerol cooking oil is used. If it is not necessary to extract an oil-soluble component with such high density, single triacylglycerol cooking oil can also be used.

FIG. 3 shows the ratio (%) of diacylglycerol and triacylglycerol in naturally occurring cooking oil.

Dietary and safe W/O type emulsifiers used in this specification include glycerol fatty acid ester, organic acid ester, cane sugar fatty acid ester, steroid lactic acid calcium, solbitan fatty acid ester, propylene glycol fatty acid ester, saponin and the like, in addition to lecithin but is not limited to these. Any substance which can effectively absorb a water-soluble component dissolved in a solvent, effectively emulsify an oil-soluble component in the digestive fluid of the small intestine and promote absorption can be used. Of these, lecithin is the most suitable emulsifier, since lecithin is essentially a bile component, and a substance for emulsifying oil and promoting its absorption.

More specifically, herbal medicines that are used in this specification include the root/stem of curcuma longa containing curcumin which is an oil-soluble component effective against liver diseases, the root/stem of spring curcuma containing much refined oil, such as sesquiterpene and the like, the root/stem of zedoary (popular name: purple curcuma) containing much refined oil and many bitter components, red pepper containing the capsanthin which is an oil-soluble component effective for fat metabolism, the leaf of Lagerstroemia speciosa pers containing the corosolic acid which is an oil-soluble component effective against diabetes, the leaf of Tithonia diversifolia (Hemsl)) containing Tagitinin C which is an oil-soluble component effective against diabetes, the leaf of Taxusbrerifolia containing Paclitaxel which is an oil-soluble component being an anticancer drug, the seed, bark and leaf of Melia azedarach L or Melia azedarach L. var. subtripinnate Miq., the seed, bark and leaf of Azadiratha indica containing the oil-soluble effective component of an anti-insect agricultural chemical traditionally used in Ayurveda of India, a medicine for diabetes, an anti-ulcer medicine, an anti-inflammation medicine and the like, the secretion of a bee, propolis, oil-soluble tetrahydrocurcumin which is an intra-living body transformation substance, oil-soluble coenzyme which is an intra-living body creation (general name: ubidecarenone C59H9004)) and the like.

In addition to the above-mentioned effective components, a part of other plants/animals, cell contents, secretions, extractions, and drug minerals or minerals which are used as medicines without being processed or become the raw materials of manufactured medicines are also called herbal medicine. In the present invention, all components contained in herbal medicines, such as effective components, which are contained and whose pharmacological effect is already regarded, other oil-soluble components, other water-soluble components and minerals are called herbal medicine components.

The first extraction method, that is, a method for extracting curcumin from the root/stem of curcuma longa is described below. By dissolving the proper amount of lecithin and/or emulsifier selected from W/O type emulsifier in oil selected from diacylglycerol and/or triacylglycerol cooking oil, mixing several percent to several tens of percent of curcuma longa root/stem powder with it and heating at 80 to 130° C., preferably 100 to 110° C. for several tens of minutes to several hours, preferably 20 to 30 minutes while stirring it, a heavy-brown muddy herbal medicine component-containing composition containing curcumin in high density is obtained.

Healthy food is obtained by enveloping the muddy oil in a capsule without any further processing. Healthy cooking oil can be obtained by absorbing/filtering the muddy oil, using a filtering assistant or filtering it with a filter press without any process.

The second extraction method, that is, a method for extracting corosolic acid from the leaf of lagerstroemia speciosa pers is described below. In the extraction of curcumin, since 3 to 6% of curcumin is contained in the dried root/stem of curcuma longa, curcumin can be extracted in a diacylglycerol solvent with high density with a single extraction operation. However, since the dried leaf of lagerstroemia speciosa pers only contains approximately 0.1 to 0.3% of corosolic acid C30H4804) and a large amount of herbal medicine is needed to extract it with high density, compared with the amount of a solvent. Therefore, it is difficult to extract it with high density with a single extraction operation. In this case, by repeating the extraction operation several times, an herbal medicine component-containing composition containing corosolic acid can be obtained with the target density.

Details are described in the third preferred embodiment later.

Next, a curcumin absorption test has been conducted on a rat. A curcumin sample was given to a rat via its mouth and discharged bile was collected for two hours. Both the curcumin in the bile and the intra-living body transformation substance of curcumin, tetrahydrocurcumin (hereinafter called “THC”) were measured by high-performance liquid chromatography (HPLC), and the sum of curcumin and THC designated as the absorbed amount of curcumin. It is because after most of curcumin absorbed in the small intestine is transformed to THC, a part of it is consumed in the liver and the remaining curcumin and THC that are not consumed is secreted in the bile, that the bile is measured. Thus, by measuring the total amount of curcumin and THC in the bile, the absorbed amount of curcumin can be calculated. In this case, the amount of intra-consumption is neglected.

The given specimen shown in FIG. 4 is described below. A specimen (1), curcumin water can be obtained by suspending 95% curcumin in water. In this case, it is assumed that the root/stem powder or tablet of curcuma longa is taken in with water. A specimen (2), curcumin-olive oil can be obtained by suspending 95% curcumin in olive oil, and it is assumed that curcuma is taken in together with fatty food. A specimen (3), an herbal medicine component-containing composition can be obtained in the second preferred embodiment of the present invention. The amount of curcumin in each given specimen is 264 mg/1 kg. FIG. 4 shows the results of the measurement.

When examining the measurement result, curcumin cannot be detected in the curcumin water (1) since curcumin can hardly be absorbed. However, for the purpose of the calculation, the amount of curcumin is designated as the lower accuracy limit of the measurement instrument, 1.6 μg.

The absorption ratio of the curcumin-olive oil (2) is 16 (=26/1.6) times as high as (1). The absorption ratio of the herbal medicine component-containing composition is 365 (=571/1.6) times as high as (1). The absorption of (3) is 21 (=571/26) times as high as (2).

According to the present invention, the herbal medicine component-containing composition obtained by the first solution means has a more bitter taste than that of curcumin extraction oil obtained without adding an emulsifier, which means that a water-soluble bitter taste component could be extracted together with oil-soluble components, such as curcumin, refined oil and the like.

The herbal medicine component-containing composition obtained by the first solution means it has been demonstrated by the curcumin absorption test of a rat that the absorption ratio of an oil-soluble component, curcumin is improved 350 times compared to that of a case where the root/stem powder or tablet of curcuma longa is taken in with water. Similarly, the absorption in the small intestine of the oil-soluble components of other herbal medicines can be promoted by the production method of the present invention.

### BRIEF DESCRIPTION OF THE DRAWINGS

FIG. 1 shows the comparison between the amount of curcumin and that of a case where the root/stem powder of curcuma longa is boiled in water.

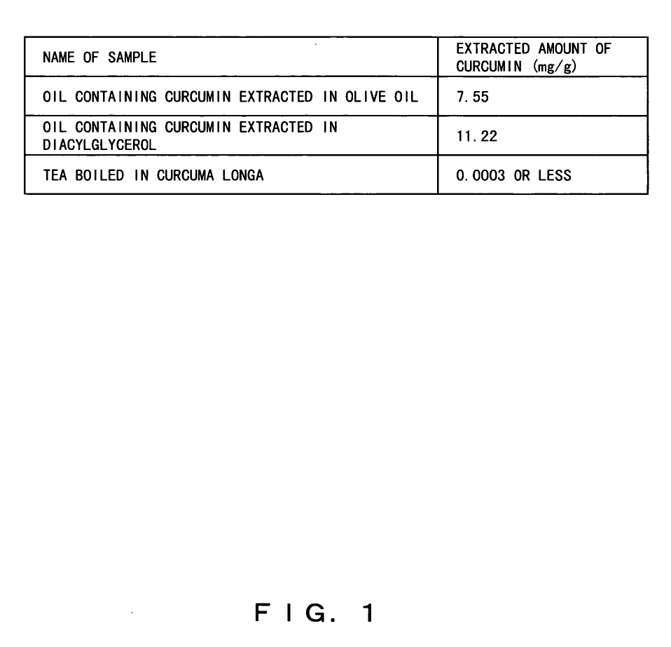

FIG. 2 shows the comparison of the extraction/non-extraction of a water-soluble component between a case where no W/O type emulsifier is added and a case where a W/O type emulsifier is added.

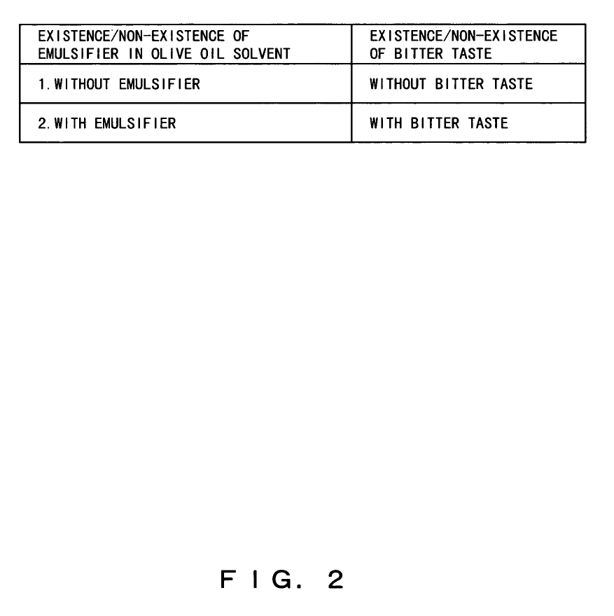

FIG. 3 shows the respective ratios (%) of naturally occurring diacylglycerol and triacylglycerol in cooking oil.

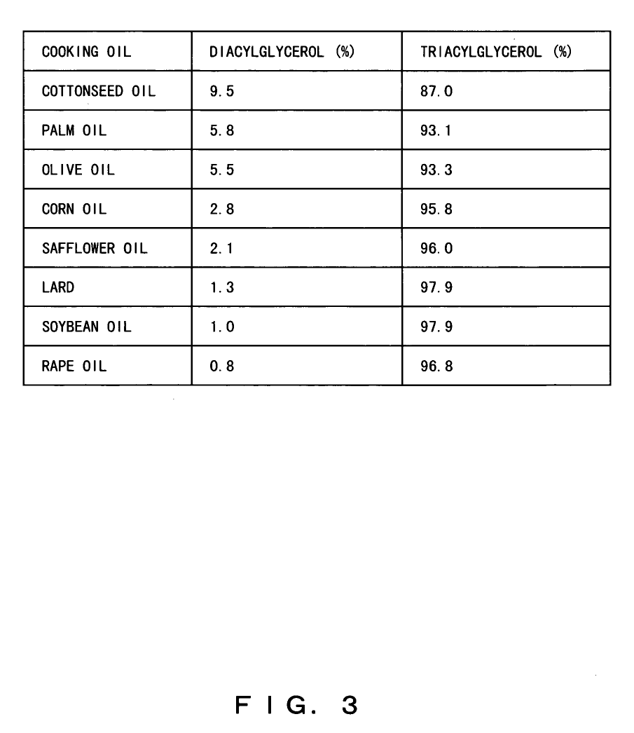

FIG. 4 explains given specimens.

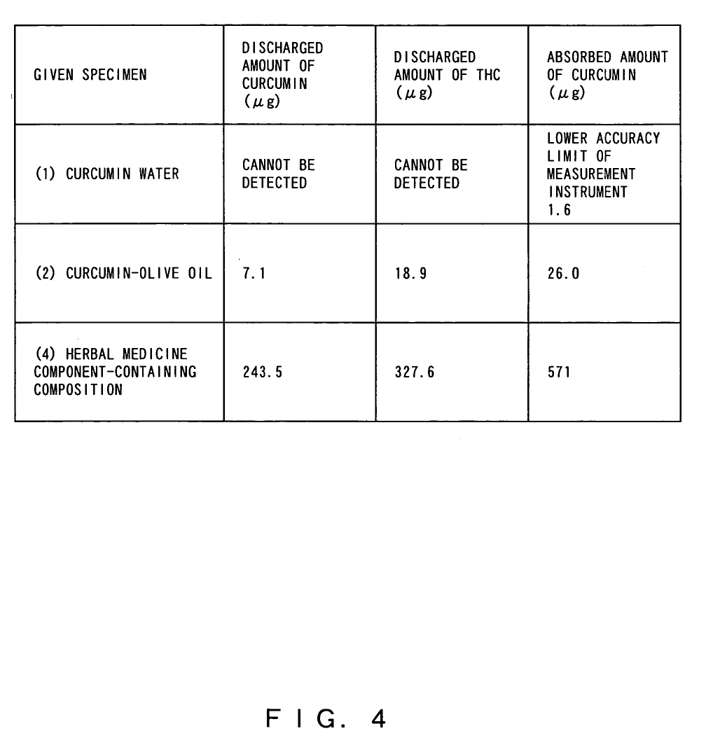

### DESCRIPTION OF THE PREFERRED EMBODIMENTS

The preferred embodiments of the present invention are described below with reference to the drawings.

In the first preferred embodiment, the powder of herbal medicines is added to solvent oil selected from diacylglycerol and/or triacylglycerol cooking oil to which lecithin and/or a W/O type emulsifier is added, and is heated and its herbal medicine component is extracted or dissolved.

5 g of lecithin is added to 100 g of diacylglycerol, is heated and is dissolved. Then, 1 g of tetrahydrocurcumin is added to this and is heated/dissolved at 90 to 100° C. for approximately 15 minutes while being stirred. An obtained transparent herbal medicine component-containing composition containing tetrahydrocurcumin is evolved with a soft capsule and is designated as healthy food.

In the second preferred embodiment, both lecithin and 2 g pf a W/O type emulsifier, 2 g of glycerol fatty acid ester are added to the total 100 g of 70 g of diacylglycerol and 30 g of olive oil and are heated/dissolved at 60° C. Then, 50 g of curcuma longa root/stem powder is added to this and is heated and extracted at 90 to 100° C. for approximately half an hour while being stirred. An obtained muddy herbal medicine component-containing composition containing curcumin with high density is evolved with a soft capsule and is designated as healthy food without any further processing.

In the third preferred embodiment, the following processes are performed.

(1) Both 5 g of lecithin and 2 g of a W/O type emulsifier, glycerol fatty acid ester are added to 100 g of diacylglycerol and are heated/dissolved at 60° C. Then, 20 g of the dried leaf powder of lagerstroemia speciosa pers containing corosolic acid with a containing ratio of 0.32% is added to this and is heated/extracted at 90 to 100° C. for half an hour. The obtained oil is absorbed/filtered using a filtering assistant, and green oil containing 0.5 mg/g is obtained with a single extraction operation.

(2) Then, similarly, 20 g of the dried leaf of lagerstroemia speciosa pers is added to 100 g of diacylglycerol using the oil obtained by the first extraction as a solvent, and is heated/extracted/filtered. Thus, with two extraction operations, green oil containing 0.9 mg/g of corosolic acid is obtained.

(3) Then, similarly, 20 g of the dried leaf of lagerstroemia speciosa pers is added to 100 g of diacylglycerol using the oil obtained by the second extraction as a solvent, and is heated/extracted/filtered. Thus, with three extraction operations, a heavy green muddy herbal medicine component-containing composition containing 1.1 mg/g of corosolic acid is obtained.

What is claimed is:

1. An herbal medicine component-containing composition containing an emulsifier, diacylglycerol and an herbal medicine component.

2. The herbal medicine component-containing composition according to claim 1, wherein the emulsifier is selected from W/O (water/oil) type emulsifiers.

3. The herbal medicine component-containing composition according to claim 1, wherein the emulsifier is lecithin.

4. The herbal medicine component-containing composition according to claim 1, 2, or 3 wherein the diacylglycerol is selected from diacylglycerol cooking oil, triacylglycerol cooking oil or their mixed oil.

5. The herbal medicine component-containing composition according to claim 1, 2, 3 or 4, wherein the herbal medicine component is composed of one or two or more types selected from curcumin. Corosolic acid, tagitinin, paclitaxel, coenzyme Q10, tetrahtdrocurcumin, an herbal medicine component of Melia azedarach L or Melia azedarach L.var. subtripinnate Miq. And an herbal medicine component of Azadiractha indica.

6. A production method of an herbal medicine component-containing composition for enabling diacylglycerol obtained by adding an emulsifier to contain an herbal medicine component.

7. The production method of a herbal medicine component-containing composition according to claim 6, wherein
the emulsifier is selected from W/O (water/oil) type emulsifiers.

8. The production method of a herbal medicine component-containing composition according to claim 6, wherein
the emulsifier is lecithin.

9. The production method of a herbal medicine component-containing composition according to claim 6, 7 or 8, wherein
the diacylglycerol is selected from diacylglycerol cooking oil, triacylglycerol cooking oil or their mixed oil.

10. The production method of a herbal medicine component-containing composition according to claim 6, 7, 8 or 9, wherein
the herbal medicine component is composed of one or two or more types selected from curcumin. Corosolic acid, tagitinin, paclitaxel, coenzyme Q10, tetrahtdrocurcumin, an herbal medicine component of Melia azedarach L or Melia azedarach L.var. subtripinnate Miq. and an herbal medicine component of Azadiractha indica.
